(19) United States
(12) Patent Application Publication
(10) Pub. No.: US 2004/0127442 A1
(43) Pub. Date: Jul. 1, 2004

(54) Oligonucleotides for treating proliferative disorders

(76) Inventors: Zhifang Zhu, Columbia, MO (US); Yuliang Cui, New York, NY (US); Qingqing Ding, Houston, TX (US); Li Li, Detroit, MI (US); Zhi Wang, Houston, TX (US)

Correspondence Address: KALOW & SPRINGUT LLP, 488 MADISON AVENUE, 19TH FLOOR, NEW YORK, NY, 10022, US

(21) Appl. No.: 10/613,390

(22) Filed: Jul. 3, 2003

Related U.S. Application Data
(60) Provisional application No. 60/400,137, filed on Aug. 1, 2002.

(51) Int. Cl. A61K 48/00

(52) U.S. Cl. 514/44

(57) ABSTRACT

The invention provides compositions and methods for treating a proliferative disorder in a subject, comprising administering a proliferation-inhibiting amount of a single-stranded oligonucleotide to the subject, wherein said single-stranded oligonucleotide is capable of binding to one or more DNA-binding proteins or RNA primers in the subject, thereby treating the proliferative disorder. The invention also provides a method for modulating transcription in a cell, comprising administering an oligonucleotide to cells, wherein the oligonucleotide consists essentially of one or more regulatory elements, wherein the one or more regulatory elements are capable of binding a DNA-binding protein.

### CROSS REFERENCE TO RELATED APPLICATIONS

This application claims priority to U.S. patent application Ser. No. 60/400,137, filed Aug. 1, 2002, which is hereby incorporated by reference into the present disclosure. This application also claims priority to the international application filed with the U.S. Patent and Trademark Office as Internatioal Receiving Office on Jun. 30, 2003, by Zhu et al., entitled “Oligonucleotides for Treating Proliferative Disorders” (attorney docket number 13552PCT; Express Mail label no. EV 279583734 US), which is hereby incorporated by reference into the present disclosure.

### BACKGROUND

Proliferative diseases, including cancers, are among the most common and pervasive disorders afflicting mammals, including man. Many of these disorders are life-shortening, and others drastically reduce the quality of life. Numerous anti-proliferative agents have been reported over the last decade. Many anti-proliferative agents have been investigated, yet few have made significant progress in bringing the world closer to a cure for cancer. Anti-cancer agents can be generally viewed as comprising five major categories: small molecules that inhibit signal transduction at the protein level, antisense therapy, siRNA/RNAi therapy, gene therapy, and immunotherapy via antibodies or surgical therapy.

Tumor genesis is commonly viewed as a process initiated by mutations in a single gene. For that reason, most anti-cancer treatments have focused on finding the mutating genes, and trying to inhibit them at the post-transcription level, usually at the protein level. This approach usually works well at the stage of in vitro studies, such as in cell culture, but often proves ineffective at the stage of in vivo studies. Tumor genesis is a multi-gene controlled process. Although the activities of one or two genes may be controlled by certain therapies, it is very likely that the tumor will find other modes of survival.

Controlling gene expression at the post-transcription level can be inefficient and ineffective. This is because the process of gene transcription is a complex one. Targeting specific proteins also limits the utility of the anti-cancer agent to a specific cancer model. This may explain why today's market is filled with a variety of anti-cancer agents. Further, blocking gene expression at the protein level can also cause harm to healthy cells because the proteins blocked by conventional anti-cancer agents are often needed by healthy cells.

Small molecule drugs can have serious adverse side effects. While blocking function of proteins produced by mutating genes, small molecule drugs can also block proteins needed by healthy cells. Since the drugs in this class figure prominently in clinical trials, the medical literature is replete with reports documenting the unpleasant side effects associated with such treatments. Gene therapy is in its infancy, and is unlikely to provide significant treatment options in the foreseeable future. Further, gene therapy is typically directed to treatment of one or two aberrant genes, thus further limiting its applicability to a wide range of proliferative disorders. Present oligonucleotide therapies, such as antisense therapy or RNA interference therapy, are, like gene therapy, directed to modulating the effects of specific aberrant genes. Immunotherapy does not offer broad treatment options, since antibodies used to fight proliferative disorders such as cancers are limited to specific antigens on specific cells. A large variety of antibodies would be necessary to treat a wide variety of disorders. Also, the costs of antibody therapy is quite high, up to thousands of dollars per dose. Likewise, cytokine therapy is very costly and can be severely debilitating to the subject undergoing such therapy.

Thus, there is a need in the art for anti-proliferative agents that preferably inhibit erratic and rapidly proliferating cells, that block the expression of over-expressed genes before their mRNAs have been synthesized, and that is not limited to a specific type of proliferative disorder, such as a specific cancer.

### SUMMARY OF INVENTION

The present invention meets these needs. The inventors herein provide methods of treating proliferative disorders wherein gene expression is modulated before mRNA is synthesized. The treatment method is particularly effective because each gene typically comprises only two alleles, although each gene can produce countless mRNA and protein molecules.

In one embodiment, the invention provides a method for treating a proliferative disorder in a subject, comprising administering a proliferation-inhibiting amount of a single-stranded oligonucleotide to the subject, wherein said single-stranded oligonucleotide is capable of binding to one or more DNA-binding proteins or RNA primers in the subject, thereby treating the proliferative disorder.

In another embodiment, the invention provides a method for modulating transcription in a cell, comprising administering an oligonucleotide to cells, wherein the oligonucleotide consists essentially of one or more regulatory elements, wherein the one or more regulatory elements are capable of binding a DNA-binding protein.

For a better understanding of the present invention together with other and further advantages and embodiments, reference is made to the following description taken in conjunction with the examples, the scope of the which is set forth in the appended claims.

### BRIEF DESCRIPTION OF THE FIGURES

The preferred embodiments of the invention have been chosen for purposes of illustration and description but are not intended to restrict the scope of the invention in any way. The preferred embodiments of certain aspects of the invention are shown in the accompanying figures, wherein:

FIG. 1 illustrates the effect of various treatments on cell growth, as measured by cell density, for breast cancer cells treated with a randomly generated 7-mer oligonucleotide, oligo #4.

FIG. 2 illustrates the survival rates of mice with murine melanoma B16 model, treated with oligo #1 and oligo #2.

FIG. 3 illustrates the survival rates of mice with leukemia ascites murine model, treated with oligo #2 and oligo #4.

### DETAILED DESCRIPTION OF THE PREFERRED EMBODIMENTS

The present invention will now be described in connection with preferred embodiments. These embodiments are presented to aid in an understanding of the present invention and are not intended, and should not be construed, to limit the invention in any way. All alternatives, modifications and equivalents that may become obvious to those of ordinary skill upon reading the disclosure are included within the spirit and scope of the present invention. Basic concepts known to those skilled in the art have not been set forth in detail.

The term “activator” includes any protein or peptide that is capable of initiating, stimulating, or maintaining transcription of a gene.

The phrase “activator-binding element” includes nucleic acid sequences that are capable of binding activators.

The term “administrating” or “administer” includes oral, rectal, topical, vaginal, intranasal, parenteral, intravenous, transdermal, and any other method of administering a pharmaceutical compound that is known in the art.

The phrase “CAAT box” includes conserved DNA sequences found typically, but not exclusively, about 75 base pairs upstream from eukaryotic transcription initiators. “CAAT boxes” are believed to be involved in binding an RNA polymerase. “CAAT boxes” typically include the nucleotide sequence caat (SEQ. ID NO. 1).

The phrase “capable of binding a DNA-binding protein” includes the ability to associate specifically with a DNA-binding protein. The phrase “associate specifically” refers to the ability of a compound, such as an oligonucleotide or a double-stranded nucleic acid, to compete away, in whole or in part, a DNA molecule bound to a DNA-binding protein.

The phrase “DNA-binding proteins” includes proteins capable of binding to DNA. The phrase includes regulatory proteins that bind to control elements of the genome, as well as single-stranded DNA-binding proteins. The phrase includes any protein molecule, for example, enzymes, enzyme subunits, or structural proteins, that can bind DNA.

An “effective amount” includes that amount of a composition that, when administered to a subject, is sufficient to effect treatment of a proliferative disorder, or sufficient to modulate transcription or DNA replication.

The phrase “GC-rich region” includes a sequence in an oligonucleotide that is at least a 5-mer, preferably at least a 6-mer, wherein the sequence has at least 5 contiguous g's and/or c's. Most preferably, a “GC-rich region” includes at least one copy of at least one of the following sequences: containing ggggcc (SEQ. ID NO. 2) and/or ccccgg (SEQ. ID NO. 3).

The phrase “human cell” includes cells derived from homo sapiens or its progeny, of whatever cell type. The phrase is meant to include haploid and diploid cells, germ cells and stem cells. The phrase is also meant to include transgenic cells, chimeric cells, products of microinjection, somatic fusion, nuclear fusion and other cells of human derivation that may contain one or more exogenous or non-human gene sequences. These exogenous sequences may be integrated into the genome of the human cell or may exist as extrachromosomal sequences, such as, for example, human cells that have been transfected with exogenous human DNA or exogenous DNA from non-human sources, such as, for example, virally-infected human cells. The phrase is meant to include transiently as well as stably transfected cell lines of human derivation. By human derivation is meant that the origin of the cells is human, without regard to subsequent manipulations that may change the genetic content of the cell line. The phrase is not meant to be limited to cells that have a normal human complement of chromosomes, since cells of human derivation may, under certain circumstances, comprise genomes that have more or less than the normal human complement of chromosomes. The phrase is meant to include cells isolated and grown or propagated in tissue culture, as well as cells in intact tissue, whether the tissue is removed or not from the human giving rise to the tissue. That is to say, the phrase “human cells” is not limited to cells grown or maintained outside the body, but is meant to include human cells inside the body as well.

The phrase “mammalian cell” includes cells derived from any mammal, including humans, of any cell type. The phrase is meant to include haploid and diploid cells, germ cells and stem cells. The phrase is also meant to include transgenic cells, chimeric cells, and other cells of human derivation that may contain one or more exogenous or non-human gene sequences. These exogenous sequences may be integrated into the genome of the mammalian cell or may exist as extrachromosomal sequences, such as, for example, mammalian cells that have been transfected with exogenous mammalian DNA or exogenous DNA from non-mammalian sources, such as, for example, virally-infected mammalian cells. The phrase is meant to include transiently as well as stably transfected cell lines of mammalian derivation. By mammalian derivation is meant that the origin of the cells is mammalian, without regard to subsequent manipulations that may change the genetic content of the cell line. The phrase is not meant to be limited to cells that have a particular mammal's normal complement of chromosomes, since cells of mammalian derivation may, under certain circumstances, comprise genomes that have more or less than the particular mammal's normal complement of chromosomes. The phrase is meant to include cells isolated and grown or propagated in tissue culture, as well as cells in intact tissue, whether the tissue is removed or not from the mammal giving rise to the tissue. That is to say, the phrase “mammalian cells” is not limited to cells grown or maintained outside the body of the mammal, but is meant to include mammalian cells inside the body as well.

The term “oligonucleotide” includes molecules that have two or more ribonucleotides and/or deoxyribonucleotides covalently bound to each other. The ribonucleotides and/or deoxyribonucleotides that comprise the oligonucleotide can include modifications of the base moiety, such as modified purines and modified pyrimidines; modifications of the ribosyl moiety, such as 2′, 3′, 4′ and/or 5′ modifications; and modifications of the internucleotide linkages in the oligonucleotide. The only limitation on the modifications of the “oligonucleotide” is that the modifications do not substantially interfere with the function of the oligonucleotide in relation to the invention. Preferably, the “oligonucleotide” retains the ability to bind to one or more DNA-binding proteins and one or more RNA primers. The base moieties of the nucleotides comprising the “oligonucleotide” can be adenine, guanine, cytosine, thymine, uracil, queuosine, inosine, hypoxanthine and the like. The base moieties can be modified to include any structure known in the art. For example, modifications can include those at exocyclic positions, and/or substitutions can be made to the atoms of the ring moieties. Modifications to the ribosyl moiety can include any structure known in the art. For example, 2′-O-alkyl modifications such as, for example, 2′-O-methyl modifications and 2′-O-methoxy ethyl modifications, 2′-alkyl modifications such as, for example, 2′-methyl modifications, 2′-halogen modifications, and the like. Preferred modifications include 2′O-methyl modifications (—O—CH3) and 2′-O-methoxy ethyl modifications (—O—CH₂CH₂—O—CH3). Internucleotide linkages can be modified to include any structure known in the art. Such modifications can include, for example, phosphorothioates, phosphorodithioates, alkyl-modified internucleotide linkages and the like. “Oligonucleotide” is meant to include all possible tautomers.

The phrase “proliferative disorder” includes diseases or disorders in which uncontrolled or abnormal cellular proliferation is implicated. Examples of proliferative disorders include cancers, including carcinomas, sarcomas, melanomas, hematopoeitic tumors of lymphoid, myeloid, or mesenchymal origin, tumors of the nervous system, and the like. Other examples include benign prostatic hyperplasia, abnormal scar formation, inflammatory bowel disease, various forms of fibrosis, arthritis, psoriasis, and the like.

The term “promoter” includes a region on a nucleotide molecule to which an RNA polymerase can bind for transcription initiation. A “promoter” is typically located upstream from the start of a structural gene.

The phrase “proliferation-inhibiting amount” includes an amount of an agent or composition, such as the compositions disclosed herein, that results in inhibition of proliferation. The inhibition of proliferation may be measured by any method known in the art. The method of measuring inhibition of proliferation will vary depending upon, for example, whether the subject treated is a human or another kind of mammal, and whether the cells are treated in situ in a mammal or in tissue culture, for example. Methods of measuring proliferation inhibition include, for example, counting cells by any method known in the art, or, for example, by measuring the area and/or volume of a tumor or a lesion. Measurement of tumors or lesions can be made directly under some circumstances, such as where the tumor or lesion is accessible or, in certain cases, where a non-human mammal is sacrificed and a tumor retrieved from the sacrificed animal. Measurement of tumors or lesions can also be made indirectly, by, for example, X-ray, MRI, CT scan, PET scan, and the like. The phrase “proliferation-inhibiting amount” includes an amount that prevents proliferation, as well as an amount that reduces proliferation. The phrase “proliferation inhibiting amount” also includes the amount of a composition that is sufficient to effect treatment of a proliferative disorder. The “proliferating inhibiting amount” can vary with the method of administration. For example, a “proliferation inhibiting amount” will be expected to be greater where treatment is systemic, as, for example, by intravenous administration, than when administration is local, such as, for example, injection directly into or in the vicinity of a tumor. Similarly, a “proliferation inhibiting amount” is expected to be lower where a cell is micro-injected with the composition than when the composition is merely included in a cell's growth medium in culture.

The phrase “randomly generated” includes any method of random generation. A particularly preferred method of making “randomly generated” oligonucleotides includes the programming of a suitable oligonucleotide synthesizing apparatus to synthesis oligonucleotides by random addition of nucleotide units.

The phrase “regulatory element” includes a nucleotide sequence implicated in regulating the expression of a gene. Examples of “regulatory elements” include operators, promoters, repressors, attenuators, enhancers, TATA boxes, CAAT boxes, GC-rich regions, AT-rich regions, and the like. “Regulatory elements” are typically, but not always, located in the 5′ direction with respect to a gene whose expression they regulate.

The phrase “regulatory protein” includes any protein capable of having an effect on transcription. Such proteins include, for example, those found associated with cellular nucleic acids in actively transcribing regions of a cell's genome.

The phrase “repressor-binding element” includes nucleotide sequences that are capable of binding proteins that can modulate transcription of genes operably linked to the nucleotide sequences.

The phrase “RNA polymerase-binding element” includes nucleotide sequences capable of binding RNA polymerases.

The phrase “RNA polymerases” includes enzymes that are capable of transcribing an RNA molecule from a DNA molecule. The phrase is meant to include all the components typically found associated with an “RNA polymerase” that is not actively transcribing, and all the components typically found associated with an “RNA polymerase” that is actively transcribing, whether in a normal, healthy cell or a cell having a disorder such as, for example, a proliferative disorder. By “actively transcribing” is meant the process of polymerizing an oligonucleotide.

The phrase “RNA primers” includes RNA molecules that are found in a cell and that act as primers for duplication of the cell's DNA.

The phrase “single-stranded nucleotide binding protein-binding elements” includes sequences that are capable of associating with single stranded DNA-binding proteins.

The term “subject” includes cells, or any organism that comprises cells, having nucleic acids. Preferably, the subject is an animal. More preferably, the subject is a human. Most preferably, the subject is a human afflicted with a proliferative disorder.

The phrase “substantially interfere” includes a reduction in function of at least 90% as compared to an oligonucleotide lacking the modification in question. For example, a modification that “substantially interferes” is one that, when the oligonucleotide is administered to a mammal in an unmodified form results in reduction of tumor growth of 50% as opposed to a mammal having the same tumor but not administered the oligonucleotide, but when administered in the modified form results in reduction of tumor growth of only 1%. Similarly, a modification that “substantially interferes” is one that, for example, when the oligonucleotide is administered to a mammal in an unmodified form results in reduction of transcription of 75% as opposed to a mammal not administered the oligonucleotide, but when administered in the modified form results in reduction of transcription of only 2%.

The phrase “TATA box,” or Hogness box (Pribnow box in prokaryotes) includes nucleotide sequences typically found about 19 to about 27 base pairs upstream from the startpoint of eukaryotic genes. The sequence is typically about 7 bases long and does not necessarily consist of the sequence tata (SEQ. ID NO. 4), but can include variations of a's and t's in an arrangement that promotes association of an RNA polymerase to the sequence. For example, a “TATA box” can have the sequence tataaaa (SEQ. ID NO.5). The phrase “TATA box” is meant to include an a- and t-rich nucleotide sequence that is capable of promoting the associating of an RNA polymerase to the sequence, and may be found outside the typical 19 to 27 base pair upstream region. Preferably, a “TATA box” has at least 5 t's and a's, preferably contiguous, such as, for example, the sequence tataa (SEQ. ID NO. 6).

The term “tautomers” includes two or more isomers of a compound comprising a keto group, for example, a nucleotide unit in an oligonucleotide, which differ by placement of a proton and the location of a double bond. For example, the keto and enol forms of a nucleotide base are referred to as tautomers. Descriptions herein of the compositions of the inventive method are meant to encompass all tautomeric forms.

The phrase “therapeutically effective amount” includes the amount of composition that, when administered to treat a disorder, is sufficient to effect treatment of the disorder. The “therapeutically effective amount” can vary with the composition, the disorder, the stage or severity of the disorder, the age, weight, and health of the subject to be treated. The “therapeutically effective amount” is expected to vary where the subject is being treated with other agents such as, for example, chemotherapeutic compounds, or when the composition is administered in conjunction with an adjuvant.

The phrase “transcription factor” includes proteins that can bind to nucleotide sequences that are control regions operably linked to genes, usually structural genes. “Transcription factors” also includes proteins that can associate with RNA polymerase and modulate transcription.

The phrase “transcription factor-binding element” includes nucleotide sequences that are capable of binding one or more transcription factors.

The terms “treatment” and “treating,” used to describe “treatment” or “treating” of a proliferative disorder, includes prevention of the disorder. Prevention of the disorder includes causing the clinical signs of the disorder not to develop. The terms are also meant to include inhibiting the disorder by, for example, slowing, stopping, or reversing the rate of progression or development of the disorder or its clinical symptoms and signs, as measured by any appropriate methodology known in the art. The terms are also meant to include partial or complete regression of the disorder or its clinical symptoms or signs. The terms also include cure, or elimination, of the disorder. “Treatment” may thus be preventive, palliative, or curative. “Treatment” is meant to include the administration of the inventive method to a subject. “Treatment” can be, for example, oral, parenteral, intravenous, topical, intranasal, rectal or intravaginal. “Treatment” can be achieved in a subject, for example, by direct injection into a region of cells afflicted with a proliferative disorder-such as a tumor-or systemic. One example is injection directly into a tumor or a region of healthy tissue adjacent to a tumor.

The term “tumor” includes a population of cells resulting from abnormal proliferation. The term “tumor” is meant to comprise benign conglomerations of cells and malignant conglomerations of cells. Benign conglomerations of cells are referred to as benign tumors. Malignant conglomerations of cells are referred to as malignant tumors.

In a first embodiment, the invention comprises method for treating a proliferative disorder in a subject, comprising administering a proliferation-inhibiting amount of a single-stranded oligonucleotide to the subject, wherein said single-stranded oligonucleotide is capable of binding to one or more DNA-binding proteins or RNA primers in the subject, thereby treating the proliferative disorder. The oligonucleotide need not be designed to hybridize, or bind, with any specific nucleic acid sequence in the cell. However, administering to a cell, together, a collection of randomly generated oligonucleotides will have the effect of competing with endogenously generated RNA primers for DNA synthesis. As a result, the randomly generated oligonucleotides will hybridize with, or bind to, the endogenously generated RNA primers and thus interfere with DNA replication. It is important to note that the exogenously added oligonucleotides need not be designed to hybridize with any specific RNA primer sequence. A sufficiently large number of randomly generated oligonucleotides, administered at once, will result in the statistical likelihood that sufficient number of RNA primers will be bound by the oligonucleotides to adversely affect DNA replication. Preferably, at least 10 to 100 randomly generated oligonucleotides of varying sequence are administered together. More preferably, at least 100 to 1,000 randomly generated oligonucleotides of varying sequence are administered together. Most preferably, more than a thousand randomly generated oligonucleotides of varying sequence are administered together.

The inventors have discovered that a proliferative disorder can be treated in a subject by administering one or more single-stranded oligonucleotides to a subject. Without wishing to be bound by any particular theory, it is believed that the single-stranded oligonucleotide(s) bind to single-stranded DNA binding proteins in the cell, effectively competing with endogenous nucleic acids for binding sites on the limited number of single-stranded DNA binding proteins in a cell. Cells have the capacity to make a limited number of single-stranded DNA binding proteins that are necessary for carrying out crucial functions in, for example, nucleic acid metabolism. For example, single-stranded DNA binding proteins are employed by the cell when replicating DNA. During replication, the cell's endogenous DNA binds to single-stranded DNA binding proteins. If exogenous single-stranded oligonucleotides are introduced into a cell, in accordance with this invention, the exogenous single-stranded oligonucleotides are expected to compete with endogenous nucleic acids for binding sites on the single-stranded DNA binding proteins. Where sufficient exogenous single-stranded oligonucleotides are administered to a subject, the subject's single-stranded DNA binding proteins will be tied up with exogenous single-stranded oligonucleotides, and be unavailable or less available to carry out functions such as, for example, assisting in DNA replication. Because an elevated level of DNA replication is necessary in cells afflicted with a proliferation disorder, administering single-stranded oligonucleotide(s) to the cells will result in decreased ability to replicate DNA and thus a decreased level of proliferation, effectively treating the proliferation disorder.

The single-stranded oligonucleotide(s) of the invention should be capable of binding to single-stranded DNA binding proteins. They need not be designed to hybridize to any particular endogenous nucleic acid sequence. Preferably, the base sequences of the oligonucleotides of the invention are randomly selected. Randomly generated oligonucleotides can be made by any method known in the art. For example, a suitable oligonucleotide synthesizer can be programmed to synthesize an oligonucleotide randomly.

Although the inventors have not found that long oligonucleotides lack antiproliferative qualities, it is known in the art that longer oligonucleotides are more difficult to administer, since longer oligonucleotides are less apt to cross cell membranes and reach the milieu in which they can exert their effects. Preferably, the oligonucleotide is from about 2 to about 40 bases in length. More preferably, the oligonucleotide is from about 5 to about 25 bases in length.

The proliferative disorder can be any proliferative disorder. Preferably, the proliferative disorder is a cancer. The invention also provides compositions, including medicaments, useful for treating proliferative disorders such as, for example, cancers.

The method can be carried out by delivering the oligonucleotide(s) of the invention in conjunction with any pharmaceutically acceptable carrier. Preferably, the pharmaceutically acceptable carrier is procaine for subcutaneous injection.

The method can be carried out with any subject. The subject can be cells or an animal, such as a mammal. If the subject is a mammal, the subject is preferably a human.

The DNA-binding proteins can be RNA polymerases, transcription factors, activators, repressors and regulatory proteins. Preferably, the DNA-binding proteins are single-stranded DNA binding proteins.

In a second embodiment, the invention provides a method for modulating transcription in a cell, comprising administering an oligonucleotide to cells, wherein the oligonucleotide consists essentially of one or more regulatory elements, wherein the one or more regulatory elements are capable of binding a DNA-binding protein.

The inventors have discovered that an oligonucleotide comprising one or more regulatory elements that are capable of binding a DNA-binding protein can be used to modulate transcription of DNA into RNA in a cell. In order for transcription in a cell to proceed normally, DNA-binding proteins bind to regulatory elements that are found, typically but not exclusively, upstream from the coding region of structural genes. At any given point in the life of a cell, a finite concentration of such proteins exists in the cell. The inventors have discovered that one or more exogenously added oligonucleotides that comprise one or more regulatory elements capable of binding a DNA-binding protein can act to compete with endogenous nucleic acid sequences, thus tying up the cell's DNA-binding proteins necessary for transcription to occur. Where a sufficient amount of exogenously added oligonucleotides comprising one or more such regulatory elements are administered to a cell, the transcription process will be slowed or stopped.

Preferably, the one or more regulatory elements is selected from the group consisting of RNA polymerase-binding elements, transcription factor-binding elements, activator-binding elements, repressor-binding elements, GC-rich regions and nucleotide binding protein-binding elements. Although the inventors have not found that long oligonucleotides lack the ability to modulate transcription, it is known in the art that longer oligonucleotides are more difficult to administer, since longer oligonucleotides are less apt to cross cell membranes and reach the milieu in which they can exert their effects. Preferably, the oligonucleotide is from about 5 to about 40 bases in length. More preferably, the oligonucleotide is from about 7 to about 25 bases in length. Preferably, the oligonucleotide is capable of intra-molecular hybridization; that is, preferably, the oligonucleotide includes self-complementary sequences. The oligonucleotide can also form concatamers.

The method can be carried out on any subject that has the ability to transcribe RNA from DNA. The subject can be an organism or a cell. Preferably, the cell is a mammalian cell, including a human cell. More preferably, the cell is a tumor cell. Where the subject is an organism, preferably it is a mammal. Where the subject is a mammal, the mammal is preferably a human.

The method can be carried out by delivering the oligonucleotide(s) of the invention in conjunction with any pharmaceutically acceptable carrier. Preferably, the pharmaceutical carrier is procaine. However, a variety of suitable pharmaceutically acceptable carriers can be used.

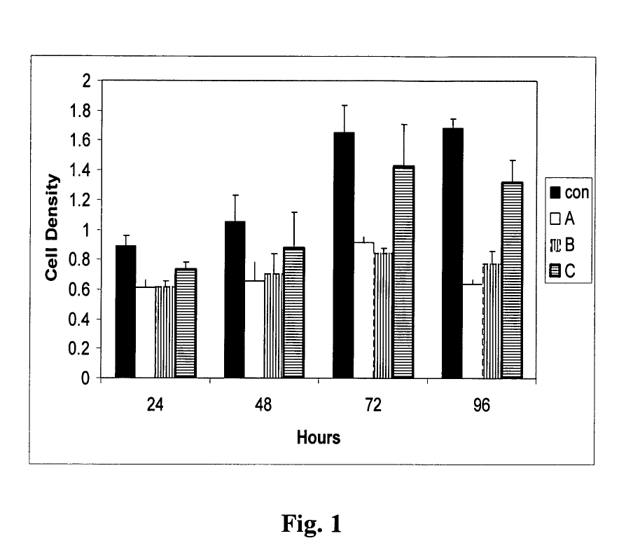

Certain advantages of the invention can be appreciated with reference to the figures. FIG. 1 depicts the effect of various treatments on tumor cell growth, as measured by cell density, for breast cancer cell growth inhibited by a randomly generated 7-mer DNA oligonucleotide, oligo #4 (Alpha DNA). Oligo #4 contains a variety of randomly generated 7-mer DNA oligonucleotides. Oligo #4 was prepared by instructing a DNA synthesizer to make, with random nucleotide sequence, a pool of 7-mers. It should be noted that the specific sequence of the oligonucleotide, as well as the specific length, is not crucial to the outcome of the experiment; any randomly-generated oligonucleotide in accordance with the invention is expected to yield similar results. SKBR3 cells were trypsinized and plated (90 microliters (1×10⁻⁴ cells)/well into 96 well plates). Ten microliters of H₂O (control) or oligo DNA were added into each well to final concentrations of 0.5×10⁻⁷ M, 0.5×10⁻⁶ M, and 10⁻⁶ M. The cells were incubated at 37° C. for 24 hrs, 48 hrs, 72 hrs and 96 hrs. The dye MTT (from Sigma) was used to assay cell viability as described by Mossman (J. Immunol. Method. 198365:55-63) with modification. Absorbance was measured at 570 nm with a multiplate reader (Bio-Rad). Growth inhibition was calculated using the formula [1−B/A]×100%, wherein A is the absorbance from the cells incubated with medium and H₂O, and B is the absorbance from the cells incubated with medium containing varying concentrations of 7-mer random oligonucleotide. FIG. 1 shows four groups: a control group, Con.; a group of cells that were administered oligo #4 at 10⁻⁶ M (one micromolar), group A; a group of cells that were administered oligo #4 at 0.5×10⁻⁶ M (half micromolar), group B; and a group of cells that were administered oligo #4 at 0.5×10⁻⁷ M (0.05 micromolar), group C. FIG. 1 establishes that oligo #4 is an effective anti-proliferation agent.

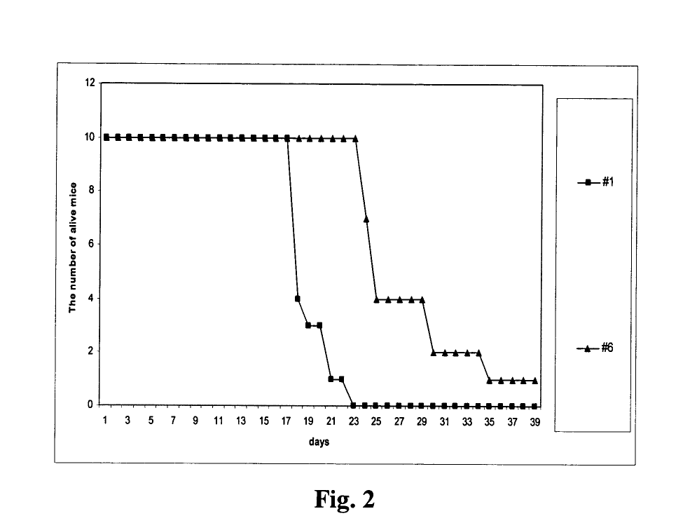

FIG. 2 illustrates the survival rates of mice with melanoma, administered varying treatments, and illustrates that the compositions and methods of the current invention are effective in treating cancer, in particular, melanoma. The experiment illustrated in FIG. 2 establishes that melanoma B16 in C57BL/6 mice responds to drug treatment in accordance with the present invention. Twenty mice (6 weeks old) were divided into two groups, having 10 mice per group. Group 1(#1) as control and Group (#6) as treatment group. On day 0 (6 weeks old), all of the mice were administered 10⁵ B 16 melanoma cancer cells/mouse intraperitoneally. From Day 1; Group 1 was administered a water injection as negative control. Group 6 was administered oligo #1, a 25 mer random oligo DNA (20 micrograms/mouse/day) (oligo #1 contains a variety of randomly generated 25-mer DNA oligonucleotides, and was prepared by instructing a DNA synthesizer to make, with random nucleotide sequence, a pool of 25-mers), and oligo #2 tattaaggggcctggccccttaata (SEQ. ID NO. 7) (20 micrograms/mouse/day), by subcutaneous injection, 5 times/week, until the mice expired. Tumor size was monitored once a week. Tumor size over 20,000 mm³ was an indicator for sacrificing.

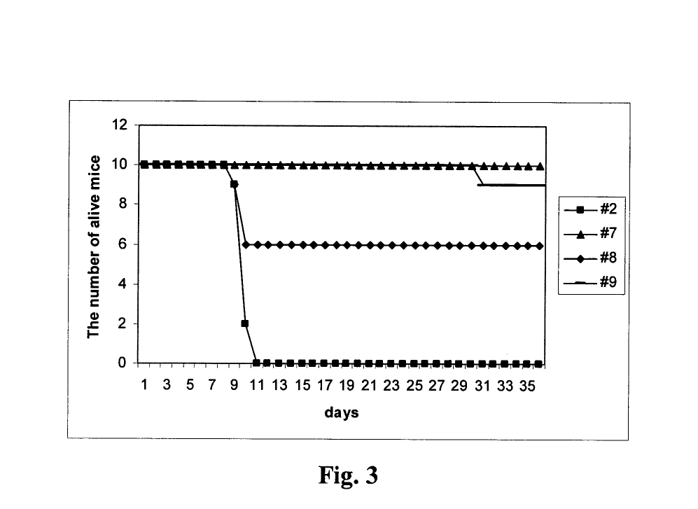

FIG. 3 illustrates the survival rates of mice with p388 leukemia ascites model, administered varying treatments. A total of 40 mice (6 weeks old) were divided into four groups. On Day 0, all four groups were intraperitoneally administered p388 leukemia cancer cells via injection (10⁶ cells/mouse). From Day 1, Group 2 (#2) were administered water as a negative control; Group 7 was administered, intravenously, oligo #2, having the sequence tattaaggggcctggcccttaata (20 micrograms/mouse/day) (SEQ. ID NO. 7), and oligo #4, which was a 7-mer random oligo DNA (20 micrograms/mouse/day); Group 8 was administered oligo #4, which was a 7-mer random oligo DNA (20 micrograms/mouse/day). Group 9 was administered Cytoxan 300 mg/kg/day (6 mg/mouse) as a positive control.

In connection with the second embodiment, oligo #2, an oligonucleotide of sequence tattaaggggcctggcccttaata (SEQ. ID NO. 7), was used. However, the invention is not limited to oligonucleotides having this particular sequence. Transcription modulation can be achieved by oligonucleotides comprising at least one of the following elements: a GC-rich region, a CAAT box, a TATA box, an AT-rich region that comprises a TATA box. The oligonucleotide(s) are preferably self-complementary. For transcription modulation, the oligonucleotide preferably comprises the sequences tattaa (SEQ. ID NO. 8), ccccgg (SEQ. ID NO. 3), and/or ggggcc (SEQ. ID NO. 2), in any order or combination. More preferably, the oligonucleotide is capable of intramolecular hybridization; that is to say, more preferably the oligonucleotide is capable of folding back and hybridizing with itself, forming a variety of 2-dimensional structures that can mimic regulatory sequences in a promoter region that form a matrix structure that transcriptional activators recognize.

Kits for administration to cells and/or subjects afflicted with a proliferative disorder comprised of the inventive compositions, with our without suitable carriers, excipients, and adjuvants, can be made. The kits can contain various combinations of the inventive compositions, in suitable containers with pre-measured doses. The kits may comprise doses in liquid, solid or gel form, and can be prepared for ready use or prepared to be reconstituted just prior to use. The kits preferably also comprise written guidelines for administration and/or treatment, and guidelines for mixing the components of the kit.

Any suitable dosage may be administered in accordance with the invention. Selection of the carrier and dose will vary depending upon the subject receiving the dose. As is known to those of skill in the art, certain factors should be considered in optimizing a dosing regimen. These factors include, for example, the specie of mammal, the mammal's body weight, the type of proliferative disorder being treated, and the health of the mammal. As a general guideline, a dosage of between about two milligrams per kilogram of body weight and about 10,000 milligrams per kilogram of body weight can be considered.

The oligonucleotide(s) of the invention can be administered alone, in mixtures, and in mixtures with other compounds or formulations for treating proliferative disorders. The dosage unit can include diluents, extenders, carriers, liposomes, and the like.

The dose can be administered in the form of a solid, gel or liquid. Dosage forms can include tablets, pills, capsules or liquid.

The dose can be administered orally, rectally, vaginally, topically, intravenously, parenterally, into or around the bone and/or its marrow.

Tablets may contain suitable binders, lubricants, diluents, disintegrating agents, coloring agents, flavoring agents, flow-inducing agents, melting agents, and the like. Tablets may be coated or uncoated to mask unpleasant taste and protect the tablet from the atmosphere and/or enteric coated to allow for selective dissolution in the digestive tract.

Capsules may be, for example, gelatin capsules comprising the inventive compositions with or without suitable carriers, such as, for example, lactose, starch, cellulose derivatives, magnesium stearate, stearic acid, and the like.

Suitable liquid dosage forms can include, for example, aqueous solutions or suspensions, pharmaceutically acceptable fats and oils, alcohols, glycols, or other organic solvents including, for example, esters, emulsions, syrups or elixirs, suspensions, solutions and/or suspensions pre-packaged or in a form to be reconstituted just prior to use. Liquid dosage forms can also include suitable solvents, preservatives, emulsifying agents, suspending agents, thickeners, diluents, sweeteners and melting agents, flavorants, coloring agents and minerals. Pharmaceutically acceptable oils include digestible oils such as, for example, olive oil, cottonseed oil, soybean oil, and the like.

The compositions of the invention may also be prepared having suitable antioxidizing agents and/or suitable preservatives.

When an anti-proliferative agent, such as a chemotherapeutic agent, is administered with the oligonucleotide(s) of the present invention, the range of dosage of the chemotherapeutic agent will generally be the same or lower than that used when the chemotherapeutic agent is administered alone. The same is true when the oligonucleotide(s) of the invention are used with an adjuvant.

The oligonucleotides of the invention can generally be administered in discrete doses at fixed time intervals, such as on a daily basis one or more times per day. As is known to those of skill in the art, it is desirable to maintain an effective concentration of the oligonucleotides in the blood of the subject being treated, or in the medium in which cells in culture are being treated. Maintaining an effective concentration of the oligonucleotides in the blood entails measuring the blood levels of the oligonucleotides in the blood at intervals following doses of varying amounts, and adjusting the dosing regimen to maintain an effective concentration in the blood. An effective concentration in the blood (or the medium in which cells in culture are being treated) corresponds to a concentration that correlates with inhibition of proliferation of cells experiencing a proliferation disorder of at least 10%. Inhibition of proliferation can be measured, for example, by estimating tumor size or proliferation activity within a tumor by any method known in the art.

According to the invention, pharmaceutical compositions or drugs comprising one or more of the therapeutic compounds described above, and a pharmaceutically acceptable carrier or excipient, may be administered to an individual predisposed or having the disease as described, orally, rectally, parenterally, systemically, intravaginally, intraperitoneally, topically (as by powders, ointments, drops or transdermal patch), bucally, or as an oral or nasal spray.

Pharmaceutical compositions used in the methods of the present invention for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions used in the present methods may also contain compounds such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drugs, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms can be made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compounds are mixed with at least one item such as a pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include items such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, may be prepared as a dry powder which may be pressurized or non-pressurized. In nonpressurized powder compositions, the active ingredients in finely divided form may be used in admixture with a larger-sized pharmaceutically acceptable inert carrier comprising particles having a size, for example, of up to 10 mm in diameter. Suitable inert carriers include sugars such as lactose. Desirably, at least 95% by weight of the particles of the active ingredient have an effective particle size in the range of 0.01 to 10 mm.

Alternatively, the composition or drugs may be pressurized and contain a compressed gas, such as nitrogen or a liquefied gas propellant. The liquefied propellant medium and indeed the total composition is preferably such that the active ingredients do not dissolve therein to any substantial extent. The pressurized composition may also contain a surface active agent. The surface active agent may be a liquid or solid non-ionic surface active agent or may be a solid anionic surface active agent. It is preferred to use the solid anionic surface active agent in the form of a sodium salt.

A further form of topical administration is to the eye. The active compounds are delivered in a pharmaceutically acceptable ophthalmic vehicle, such that the compounds are maintained in contact with the ocular surface for a sufficient time period to allow the compounds to penetrate the corneal and internal regions of the eye, as for example the anterior chamber, posterior chamber, vitreous body, aqueous humor, vitreous humor, cornea, iris/cilary, lens, choroid/retina and sclera. The pharmaceutically acceptable ophthalmic vehicle may, for example, be an ointment, vegetable oil or an encapsulating material.

Compositions for rectal or vaginal administration are preferably suppositories which can be prepared by mixing the active compounds with suitable non-irritating excipients or carriers such as cocoa butter, polyethylene glycol or a suppository wax which are solid at room temperature but liquid at body temperature and therefore melt in the rectum or vaginal cavity and release the drugs.

The compositions used in the methods of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form, in addition to one or more of the active compounds described above, can contain stabilizers, preservatives, excipients and the like. The preferred lipids are the phospholipids and the phosphatidyl cholines (lecithins), both natural and synthetic.

The above methods of the present invention can be used in vivo, in vitro, in situ and ex vivo, for example, in living mammals as well as in cultured tissue, organ or cellular systems. Mammals include, for example, humans, as well as pet animals such as dogs and cats, laboratory animals, such as rats and mice, hamsters and farm animals, such as, for example, horses, chickens, pigs, and cows. Tissues, as used herein, are an aggregation of similarly specialized cells which together perform certain special functions.

The methods and compositions of the present invention can be administered in conjunction with know treatments for proliferative disorders and methods of modulating transcription. For example, the methods and compositions can be used together with any known chemotherapeutic agent known in the art. Chemotherapeutic agents include DNA-targeting agents such as cisplatin, carboplatin, idarubicin, mitoxantrone, cyclophosphamides, altretamine, bleomycin, dactinomycin, doxorubicin (including liposomal doxorubicin), etoposide, teniposide, plicamycin; taxol; alkylating agents such as, for example, chloorambucil, cyclophoosphamide, isofamide, mechlorethamine, melphalan, uracil mustards; aziridines such as thiotepa; busulfan, carmustine, lomustine, strepozocin; mitomycin, procabazine, dacarbazine, methotrexate, trimetrexate, fluorouracil, fluorodeoxyuridine, floxuridine, mercaptopurine, pentostatin, 6-thioguanine, cyarabine, fludarabine, hydroxyurea, colchicine, vincristine, vinblastine, paclitaxel, cytoxam, steroids, modified steroids, estrogens, modified estrogens, progestins, modified progestins, hydorxyprogesterone caproate, medroxyprogeeterone, megestrol, androgens and modified androgens, testosterone and salts thereof, fluoxymestrone, methyltestosterone, adrenal corticosteroids, cortisol, hydrocortisone, prednisone, dexamethasone, methylprednisolone, prednisolone, leutinizing hormone, leuprolide acetate, goserelin acetate, tamoxifen, flutamide, mitoxane, aminoglutethimide, asparaginase, benzimidazoles, griseofulvin, interferons, interleukins, antimetabolites, protein kinase inhibitors, protease inhibitors, antibodies such as, for example, herceptin, anti-neoplastic agents, and the like. This list is not meant to be exhaustive, but illustrative only. Further, the compositions and methods can be employed in conjunction with non-chemotherapeutic methods as well, including, for example, radiation therapy and surgery.

Having now generally described the invention, the invention may be more readily understood through reference to the following examples, which are provided by way of illustration and are not intended to limit the present invention unless specified.

### EXAMPLES

The following examples are intended to explain the invention further.

### Growth Inhibition of Tumor Cells

SKBR3 cells, human breast cancer cells that over-express the Her gene, were trypsinized and plated at 90 microliters (1×10⁴ cells)/well into 96 well plates. Ten microliters of H₂O (control) or oligo DNA (from Alpha DNA, Canada)] were added into each well to final concentrations of 0.5×10⁻⁷ M, 0.5×10⁻⁶ M and 10⁻⁶ M. The cells were incubated at 37° C. for 24 hrs, 48 hrs, 72 hrs and 96 hrs. The dye MTT (from Sigma) was used to assays cell viability as described by Mossman (J. Immunol. Method. 198365:55-63) with modification. Absorbance was measured at 570 nm with a multi-plate reader. Growth inhibition was calculated using the formula [1−B/A]×100%, wherein A is the absorbance of cells incubated with medium and water, and B is the absorbance of cells incubated with medium containing varying concentrations of oligonucleotides administered.

### Microarray Processing

SKBR3 cells were incubated with oligo-nucleotide DNA2 (oligo #2), having the sequence tattaaggggcctggccccttaata (SEQ. ID NO. 7) at a concentration of 10⁻⁶ M for 48 hrs. Total RNA was isolated from the cells using TRIzol reagent (Invitrogen). An Agilent human cDNA catalog microarray was examined using the SKBR3 RNA. Up to 13,000 human genes were examined. Among these genes, 1,100 genes were found to have been up-regulated, whereas 1,000 genes were found to have been down regulated. Since gene expression in living cells is a dynamic process, the expression pattern is expected to change over time. The inventors expected that exogenous transcription activator-binding DNA, the oligonucleotide designated oligo DNA 2 (oligo #2), would interfere with endogenous gene expression patterns. The inventors hypothesized that the exogenously added oligonucleotide would inhibit over-expression of genes that may be responsible for initiation or maintenance of pathological states. The inventors also hypothesized that suppressed genes would be up-regulated, possibly contributing to gain of normal function. Because the immune systems of cancer patients exhibit functional deficits, the inventors expected to observe changes in regulation of immune response genes. The inventors found that, of 25 immune response genes examined, 19 were up-regulated in the SKBR3 cells.

Melanoma B16 in C57BL/6 Mice: Response to Drug Treatment

Twenty mice (6 weeks old) were divided into two groups, having 10 mice per group. Group 1 (#1) was a control group; Group (#6) was a treatment group. On Day 0, (6 weeks old) all of the mice were administered 10⁵ B 16 melanoma cancer cells/mouse intraperitoneally. From Day 1; Groups 1, was administered water injections as negative control. Group 6 was administered oligo #1, a 25-mer random oligo DNA (20 micrograms/mouse/day), and oligo #2 tattaaggggcctggccccttaata (SEQ. ID NO. 7) (20 micrograms/mouse/day), by subcutaneous injection, 5 times/week, until the mice expired. The mixture of oligo #1 (20 micrograms/mouse/day) and oligo #2 (20 micrograms/mouse/day) were dissolved in 1% procaine, and was administered by subcutaneous injection. Group 1, as negative control, was administered water by injection. Days 8-14, 15-21, 22-28, 29-35, 36-42, and 43-49 followed the same schedule as Days 1-7. Tumor size was monitored once a week. Tumor size over 20,000 mm³ was an indicator for sacrificing. The mean survival times of all groups were calculated, and the results were expressed as mean survival of treated mice/mean survival of control mice (T/C)×100%. The results establish a T/C=150, which shows modest activity. The dosing schedule is illustrated in Table 1.

TABLE 1
Injection Schedule for Oligo #1 and Oligo #2

| | Day Number | | | | | | | |
|---|---|---|---|---|---|---|---|---|
| Group | 0 | 1 | 2 | 3 | 4 | 5 | 6 | 7 |
| 1 | B16, 10⁵ I.p. | H₂O | H₂O | H₂O | H₂O | H₂O | | |
| 6 | B16, 10⁵ I.p. | #1, 2 | #1, 2 | #1, 2 | #1, 2 | #1, 2 | | |

#1, 2 refer to oligo #1 and oligo #2

Survival Test in Leukemia (p388)Ascites Mice Model

A total of 40 mice (6 weeks old) were divided into 4 groups. On Day 0, all 4 groups of the mice were administered p388 leukemia cancer cells by intraperitoneal injection (10⁶ cells/mouse). From day 1, Group 2 were administered water intravenously as negative controls. Group 7 received oligo 2 (tattaaggggcctggccccttaata, SEQ. ID NO. 7) intravenously at 20 micrograms/mouse/day and oligo 4 (a 7-mer random oligo DNA) intravenously at 20 micrograms/mouse/day. Group 8 was administered oligo 4, a 7-mer random oligo DNA at 20 micrograms/mouse/day. Group 9 received Cytoxan at a dose of 300 milligrams/kg/day (6 mg/mouse), as positive controls. Days 8-14, 15,-21, 22-28, 29-35, 36-42, and 43-49 followed the same schedule as Days 1-7. The dosing schedule is illustrated in Table 2.

TABLE 2
Oligo #1, #2, and #4 Treatment Schedule
for p388 Murine Leukemia Ascites Model

| | Day | | | | | | | |
|---|---|---|---|---|---|---|---|---|
| Group | 0 | 1 | 2 | 3 | 4 | 5 | 6 | 7 |
| 2 | p388 I.p. | H₂O | H₂O | H₂O | H₂O | H₂O | H₂O | H₂O |
| 7 | p388 I.p. | #2 + #4 | #2 + #4 | #2 + #4 | #2 + #4 | #2 + #4 | #2 + #4 | #2 + #4 |
| 8 | p388 I.p. | #4 | #4 | #4 | #4 | #4 | #4 | #4 |
| 9 | p388 I.p. | cytxn | cytxn | cytxn | cytxn | cytxn | cytxn | cytxn |

#2: 25-mer tattaaggggcctggccccttaata
4: 7-mer random oligo DNA
Group7 got The mixture #2 1 mg/kg/day, and #4, 1 mg/kg/day; is injected i.v.
Group 8 got #4 1 mg/kg/day i.v.
Group 9 is positive control, cytoxan 300 mg/kg; I.p.; qdxl
Group 2 is negative controls, get H2O injection
Cytxn = cytoxan

The results show that oligo oligo #2, tattaaggggcctggcccttaata (SEQ. ID NO. 7), at a dosage of 1 mg/kg/day i.v with 7-mer random oligo DNA at 1 mg/kg/day i.v attained perfect results, up to 36 days after cancer cell injection, with a 100% survival rate. The 7-mer random oligo #4, at 1 mg/kg/day i.v., attained a 60% survival rate.

These in vivo results establish that the 7-mer random oligo DNA can interfere with DNA synthesis, and the 25-mer oligo DNA tattaaggggcctggccccttaata (SEQ. ID NO. 7) can interfere with RNA transcription. The 7-mer random oligo DNA does not achieve results as good as results using both the 7-mer random oligo DNA plus the 25-mer tattaaggggcctggccccttaata (SEQ. ID NO. 7). One possibility is that the dose of the 7-mer, when used alone, is not high enough. A second possibility is that modulating only DNA synthesis is not as effective as modulating both DNA synthesis and RNA transcription.

While the invention has been described in connection with specific embodiments, it will be understood that it is capable of further modifications and this application is intended to cover any variations, uses, or adaptations of the invention following, in general, the principles of the invention and including such departure from the present disclosure as come within known or customary practice within the art to which the invention pertains and as may be applied to the essential features described herein and as follows in the scope of the appended claims.

What is claimed is:

1. A method for treating a proliferative disorder in a subject, comprising administering a proliferation-inhibiting amount of a single-stranded oligonucleotide to the subject, wherein said single-stranded oligonucleotide is capable of binding to one or more DNA-binding proteins or RNA primers in the subject, thereby treating the proliferative disorder.

2. A method according to claim 1, wherein the single-stranded oligonucleotide is randomly generated.

3. A method according to claim 1, wherein the single-stranded oligonucleotide is from about 2 to about 40 bases in length.

4. A method according to claim 3, wherein the single-stranded oligonucleotide is from about 7 to about 25 bases in length.

5. A method according to claim 1, wherein the proliferative disorder is a cancer.

6. A method according to claim 5, wherein the cancer is randomly selected from the group consisting of leukemia, lung cancer and melanoma.

7. A method according to claim 1, wherein the single-stranded oligonucleotide is administered with a pharmaceutically acceptable carrier.

8. A method according to claim 7, wherein the pharmaceutically acceptable carrier is procaine.

9. A method according to claim 1, wherein the subject is a human.

10. A method according to claim 1, wherein the DNA-binding proteins are single-stranded DNA binding proteins.

11. A method according to claim 1, wherein the DNA-binding proteins are selected from the group consisting of RNA polymerases, transcription factors, activators, repressors and regulatory proteins.

12. A method for modulating transcription in a cell, comprising administering an oligonucleotide to cells, wherein the oligonucleotide consists essentially of one or more regulatory elements, wherein the one or more regulatory elements are capable of binding a DNA-binding protein.

13. A method according to claim 12, wherein the one or more regulatory elements is selected from the group consisting of RNA polymerase-binding elements, transcription factor-binding elements, activator-binding elements, repressor-binding elements, GC-rich regions and single-stranded nucleotide binding protein-binding elements.

14. A method according to claim 12, wherein the oligonucleotide is from about 5 to about 40 bases in length.

15. A method according to claim 14, wherein the oligonucleotide is from about 7 to about 25 bases in length.

16. A method according to claim 12, wherein the cell is a mammalian cell.

17. A method according to claim 12, wherein the cell is a tumor cell.

18. A method according to claim 12, wherein the cell is a human cell.

19. A method according to claim 12, wherein the oligonucleotide is administered with a pharmaceutically acceptable carrier.

20. A method according to claim 19, wherein the pharmaceutically acceptable carrier is procaine for subcutaneous injection.

21. A method according to claim 12, wherein the oligonucleotide comprises the sequence tattaaggggcctggccccttaata (SEQ. ID NO. 7).


SEQUENCE LISTING (1 sequence)

SEQ ID NO: 1 - DNA, 25 bp, artificial sequence
  synthetic primer
tattaaggggcctggccccttaata